(19) United States
(12) Patent Application Publication
(10) Pub. No.: US 2022/0282123 A1
(43) Pub. Date: Sep. 8, 2022

(54) METHOD FOR PRODUCING BONE GELATINE, AND PRODUCED BONE GELATINE

(71) Applicant: GELITA AG, Eberbach (DE)

(72) Inventor: Ralf PÖRSCHKE, Eschweiler (DE)

(21) Appl. No.: 17/736,396

(22) Filed: May 4, 2022

Related U.S. Application Data
(63) Continuation of application No. PCT/EP2020/077094, filed on Sep. 28, 2020.

(30) Foreign Application Priority Data
Nov. 8, 2019 (DE) 102019130197.1

(51) Int. Cl. C09H 3/00; A23J 1/10; A23J 3/06; C08H 1/06

(52) U.S. Cl. CPC C09H 3/00; A23J 1/10; A23J 3/06; C08H 1/06

(57) ABSTRACT

The present invention relates to a method for producing bone gelatine having an isoelectric point of less than 6, comprising the following steps: a) providing bones of vertebrates; b) mechanically crushing the bones to a particle size of less than 1 500 μm, preferably less than 500 μm, more preferably less than 300 μm; c) extracting the crushed bones using an aqueous medium at a temperature of from 100 to 140° C., preferably from 120 to 130° C., for a period of from 0.5 to 10 min, preferably 1 to 5 min, more preferably 1 to 3 min; d) separating off the aqueous gelatine solution from the crushed bones; and e) drying the aqueous gelatine solution in order to obtain the bone gelatine having an isoelectric point of less than 6, wherein the method does not comprise liming of the bones with a base, and wherein the bones provided in step a) have not undergone liming. The invention further relates to bone gelatine having an isoelectric point of less than 6, produced by this method.

### CROSS-REFERENCE TO RELATED APPLICATIONS

This patent application is a continuation of International Patent Application No. PCT/EP2020/077094, filed Sep. 28, 2020, which claims the benefit of German Patent Application No. 10 2019 130 197.1, filed Nov. 8, 2019, which are each incorporated by reference in its entirety herein.

### FIELD OF THE INVENTION

Obtaining gelatine from animal bones has long been known. Like other starting materials for producing gelatine (such as animal skin), bones contain high proportions of the structural protein collagen, in particular type I collagen.

The typical method used on an industrial scale for producing bone gelatine is a multistep procedure that comprises as the essential steps the demineralisation of the bones in a strongly acidic medium (maceration) and a subsequent treatment in a strongly alkaline medium (liming), in order then to be able to extract the gelatine at elevated temperature (typically between 50 and 100° C.) in a plurality of steps (see R Schrieber and H Gareis: Gelatin Handbook, 2007, Section 2.2.5).

During maceration, the roughly ground bones are treated for a period of approximately a week in a counter-current process with dilute hydrochloric acid, in order to elute the mineral components (calcium carbonate and calcium phosphate) from the bone tissue (see Gelatin Handbook, Section 2.2.1.1). The product obtained by this process is called ossein. A cost factor of relevance in maceration is the required cooling because of the exothermic reaction of hydrochloric acid with the calcium minerals. A further disadvantage is the high chloride load in the waste water.

The subsequent liming of the ossein is necessary to enable effective extraction of the gelatine. Typically, the liming process comprises a treatment with a calcium hydroxide suspension (pH value of more than 12) for a period of several months (see Gelatin Handbook, Section 2.2.4.1). Although the treatment time may be shortened by using stronger alkalis (for example to a few days when sodium hydroxide is used), this results in a loss in yield.

The method described above gives type B bone gelatine, which is characterised by an isoelectric point (IEP) of less than 5.6, typically in the range of 4.8 to 5.5. The IEP corresponds to the pH value at which the polypeptide chains of the gelatine (or the collagen peptides produced therefrom) have a neutral overall charge. The relatively low IEP of type B gelatine results from the fact that during liming the amino acids asparagine and glutamine are converted almost entirely to aspartic acid and glutamic acid respectively.

For this reason, the production of type B bone gelatine gives rise to relatively high costs in terms of tying up capital, because of the long treatment times and/or because of the high consumption of chemicals.

Also known is the production of type A bone gelatine, in which the ossein is extracted in the acidic medium, without liming. Type A gelatines have a high IEP, greater than 6, in the case of type A bone gelatine typically in the range of between 6 and 8 (in the case of pork rind gelatine in the range of from 8 to 9). Because of its lower viscosity at the same gel strength than type B gelatine, however, type A gelatine is only suitable for a few areas of application. Moreover, type A bone gelatine is in competition with pork rind gelatine, which can be produced at considerably less expense.

### BRIEF SUMMARY OF THE INVENTION

The present invention relates to a method for producing bone gelatine having an isoelectric point of less than 6.

The invention further relates to a bone gelatine that is produced by this method.

### DETAILED DESCRIPTION OF THE INVENTION

The object of the invention is to propose a simple and low-cost method for producing bone gelatine having a low isoelectric point.

This object is achieved according to the invention with the method of the type mentioned in the introduction in that it comprises the following steps:

a) providing bones of vertebrates;

b) mechanically crushing the bones to a particle size of less than 1 500 μm, preferably less than 500 μm, more preferably less than 300 μm;

c) extracting the crushed bones using an aqueous medium at a temperature of from 100° C. to 140° C., preferably from 120° C. to 130° C., for a period of from 0.5 to 10 min, preferably 1 to 5 min, more preferably 1 to 3 min;

d) separating off the aqueous gelatine solution from the crushed bones; and

e) drying the aqueous gelatine solution in order to obtain the bone gelatine having an isoelectric point of less than 6,

wherein the method does not comprise liming of the bones with a base, and wherein the bones provided in step a) have not undergone liming.

Within the context of the invention, it was surprisingly found that gelatine from bones can be produced by this simple method in relatively few process steps if the corresponding method parameters are observed. In this case, the bone gelatine produced according to the invention has a low IEP of less than 6, and is thus suitable for the typical areas of application of type B gelatine.

The method according to the invention explicitly dispenses with liming of the bones, which in the extreme case takes several months, but at least several days. This alone significantly reduces the time and energy requirement and the waste water pollution of the method according to the invention. In the context of the present description, the term “liming” is understood to mean a treatment with a base at a pH value of greater than 12.

Maceration of the bones beforehand is possible within the context of the production method according to the invention, but is not fundamentally necessary. For this reason, preferably the method according to the invention does not comprise maceration of the bones with an acid, and the bones provided in step a) have preferably not undergone maceration. This also reduces the time requirement and the costs of the method. In the context of the present description, the term “maceration” is understood to mean a treatment with an acid at a pH value of less than 1.

As the starting material for the method according to the invention, there may be used in principle bones of any vertebrates, thus including those of birds or fish. Preferably, however, the method is carried out with bones of mammals, in particular bones of bovines.

It is favourable if the bones are cleaned before being crushed, in particular being degreased. Cleaning the starting material favours efficient extraction in step c), and makes it possible to produce high-quality bone gelatine.

Preferably, the cleaning of the bones comprises a treatment with one or more enzymes, preferably with proteases and/or lipases. While lipases serve for degreasing, non-collagenous proteins can be broken down and removed using proteases. The proteases hydrolyse the collagen to an only negligible extent before the bones are appropriately crushed.

Before crushing, the bones favourably have a fat content of less than 4 weight %, preferably less than 1 weight %, more preferably less than 0.5 weight %.

The mechanical crushing of the preferably cleaned bones to give a particle size of less than 1 500 μm is an essential feature of the method according to the invention. The small particle size enables the direct extraction of gelatine from the bone material without the need for the pre-treatments known from the prior art, such as maceration or liming. The mechanical crushing may comprise dry grinding or wet grinding of the bones. Wet grinding is preferably performed in the aqueous medium for the extraction.

The extraction in the method according to the invention is preferably carried out with a content of crushed bones by weight in the aqueous medium of from 0.05 to 0.5 kg/1, preferably from 0.1 to 0.3 kg/1, more preferably from 0.15 to 0.2 kg/l.

In the method according to the invention, extraction of the crushed bones is performed at an elevated temperature of from 100 to 140° C., that is to say that the extraction is substantially brought about by the input of thermal energy. Optionally, the extraction may be further accelerated and/or intensified by an additional input of energy by means of cavitation, for example by ultrasound or a high-pressure homogeniser. Another possibility is to apply AC electrical fields during the extraction.

It is favourable if the extraction is carried out at a pH value in the range of from 7.5 to 9, preferably from 8 to 8.5. By adjusting the pH value during the extraction, it is possible to influence the isoelectric point of the bone gelatine produced. According to the invention, this is less than 6, preferably in the range of from 5.2 to 5.6.

Before the extraction, the pH value is adjusted in particular by adding a base, preferably by adding a sodium hydroxide or calcium hydroxide solution.

In a preferred embodiment of the invention, once the aqueous gelatine solution has been separated off, the crushed bones undergo steps c) and d) up to two further times. A multiple extraction can increase the yield of gelatine. Here, for each extraction step the relatively short extraction time of at most 10 min in step c) of the method according to the invention is sufficient.

Preferably, separating off the aqueous gelatine solution from the crushed bones comprises a filtration, in particular a membrane filtration. This allows even the smallest particles of crushed bones and other solids to be removed.

After filtration, the aqueous gelatine solution (where appropriate after combining the result of a plurality of extractions) may preferably undergo an ion exchange procedure, in particular salt removal.

The drying of the aqueous gelatine solution in order to obtain the bone gelatine having an IEP of less than 6 may be performed by various methods, which are known per se from the prior art. Typically, in a first step a concentration is carried out using evaporators and/or ultrafiltration. Finally, the gelatine may be dried over heated rollers or in convection driers after gelation, until it has a desired water content of typically 10 to 12%.

The present invention also relates to a bone gelatine having an isoelectric point of less than 6 that is produced by the method according to the invention.

Preferably, the bone gelatine according to the invention has an isoelectric point of from 5.2 to 5.6.

The bone gelatine according to the invention can be used in all the usual areas of application of type B gelatine. Particularly advantageously, the bone gelatine according to the invention is suitable for making soft capsules of gelatine.

These and further advantages of the invention become apparent from the example described below.

### Example 1

### Example: Production of Bone Gelatine on a Laboratory Scale

735 g of water was added to 165 g of bone powder (d50<350 μm; d90<700 μm) from bovine bones. The pH value of the suspension formed was adjusted to a value of 8.5 using sodium hydroxide solution. Then, the suspension was heated in a microwave oven to between 120 and 127° C. for approximately 1 min, with stirring.

After cooling (for approximately 20 min) to 95° C., it was possible to separate off the aqueous solution of the gelatine extracted from the bone powder from the solid components. The separated solids underwent the procedure described once more, and the combined gelatine solutions were worked up by filtration using a sheet filter.

After a complete ion exchange, an aqueous gelatine solution having a conductivity of 2.1 μS and a pH value of 5.5 was obtained. This pH value corresponds approximately to the isoelectric point of gelatine.

The yield of bone gelatine after this two-stage extraction is 16 weight % relative to the bone powder used.

1. A method for producing bone gelatine having an isoelectric point of less than 6, comprising the following steps:
a) providing bones of vertebrates;
b) mechanically crushing the bones to a particle size of less than 1 500 μm;
c) extracting the crushed bones using an aqueous medium at a temperature of from 100 to 140° C., for a period of from 0.5 to 10 min;
d) separating off the aqueous gelatine solution from the crushed bones; and
e) drying the aqueous gelatine solution in order to obtain the bone gelatine having an isoelectric point of less than 6,
wherein the method does not comprise liming of the bones with a base, and wherein the bones provided in step a) have not undergone liming.

2. The method according to claim 1, wherein the method does not comprise maceration of the bones with an acid, and wherein the bones provided in step a) have not undergone maceration.

3. The method according to claim 1, wherein the bones come from mammals.

4. The method according to claim 1, wherein the bones are cleaned before being crushed.

5. The method according to claim 4,
wherein the cleaning of the bones comprises a treatment with one or more enzymes.

6. The method according to claim 1, wherein, before crushing, the bones have a fat content of less than 4 weight %.

7. The method according to claim 1, wherein the mechanical crushing comprises dry grinding or wet grinding of the bones.

8. The method according to claim 1, wherein the extraction is carried out with a content of crushed bones by weight in the aqueous medium of from 0.05 to 0.5 kg/1.

9. The method according to claim 1, wherein the extraction is carried out at a pH value in the range of from 7.5 to 9.

10. The method according to claim 9,
wherein, before extraction, the pH value is adjusted by adding a base.

11. The method according to claim 1, wherein, once the aqueous gelatine solution has been separated off, the crushed bones undergo steps c) and d) up to two further times.

12. The method according to claim 1, wherein separating off the aqueous gelatine solution comprises a filtration.

13. The method according to claim 1, wherein drying the gelatine solution comprises roller drying or belt drying.

14. A bone gelatine having an isoelectric point of less than 6, produced by the method according to claim 1.

15. The bone gelatine according to claim 14, having an isoelectric point of from 5.2 to 5.6.
